(19) United States
(12) Patent Application Publication
(10) Pub. No.: US 2025/0352612 A1
(43) Pub. Date: Nov. 20, 2025

(54) PHARMACEUTICAL AGENT TO PREVENT THE ADVERSE EFFECTS OF ROTAVIRUS NSP4 PROTEIN

(71) Applicants: AFYONKARAHISAR SAGLIK BILIMLERI ÜNIVERSITESI REKTÖRLÜGÜ, Afyonkarahisar (TR); EGE ÜNIVERSITESI IDARI VE MALI ISLER DAIRE BASK., Izmir (TR)

(72) Inventors: Merve SEN, Afyonkarahisar (TR); Hüseyin TASLI, Bornova/Izmir (TR); Sefa ÇELIK, Afyonkarahisar (TR)

(21) Appl. No.: 18/866,239

(22) PCT Filed: Apr. 4, 2023
(86) PCT No.: PCT/TR2023/050315

§ 371 Date: Nov. 15, 2024

(30) Foreign Application Priority Data
May 17, 2022 (TR) 2022/007962

(51) Int. Cl. A61K 38/16; A61P 29/00

(52) U.S. Cl. CPC A61K 38/164; A61P 29/00

(57) ABSTRACT

Disclosed is a pharmaceutical agent for the prevention and/or elimination of the adverse effects of the rotavirus NSP4 viral protene in cells and/or organisms using the Lactobacillus plantarum bacteriocin PlnA.

### TECHNICAL FIELD

The invention relates to a pharmaceutical agent for the prevention and/or elimination of the adverse effects caused by the rotavirus NSP4 protein.

In particular, the invention relates to a pharmaceutical agent for the prevention and/or elimination of the adverse effects of the rotavirus NSP4 viral protene in cells and/or organisms using the Lactobacillus plantarum bacteriocin PlnA.

### STATE OF THE ART

Rotavirus is the most common viral gastroenteritis agent, especially in children, and causes severe cases of diarrhea. In a report published by the World Health Organization (WHO) in April 2016, it was stated that 528,000 children died in 2000 and 215,000 in 2013 due to rotavirus infection (World Health Organization [WHO], 2019). Rotavirus infections, which have become a global health problem, accounted for 38% of all diarrhea cases in 2013, and many children are still at risk due to rotavirus. According to a study by Tate et al. in 2016, rotavirus infections were responsible for the deaths of 215,000 children aged 5 years and under, with hundreds of thousands of hospitalizations.

Rotavirus is an enteric virus that causes diarrhea mostly in children and newborns and can cause infection by fecal-oral ingestion of more than 100 virus particles. In rotavirus infections, malabsorption, changes in cell permeability and chloride secretion, enterocyte damage and diarrhea occur with the effects of NSP4 (Nonstructural protein 4) viral protein. Rotavirus shows enterocyte involvement and cell death after infection leads to proliferation of secretory crypt cells and loss of fluid and electrolytes in the lumen. The decrease in digestive enzymes along with enterocytes causes carbohydrate malabsorption and diarrhea. This diarrhea caused by the disturbance of osmotic balance is accompanied by secrotoric diarrhea caused by the NSP4 protein affecting cellular permeability and calcium mobilization.

Therefore, it poses a great threat to human health, especially to newborns and children, and causes deaths.

The endoplasmic reticulum (ER) functions as an intracellular calcium store and contributes to the synthesis and post-translational modifications of secretory and membrane proteins. Disruption of ER function for various reasons leads to ER stress and the unfolded protein response (UPR). The primary goal of this response is to compensate for the stress and ensure cell survival. However, under conditions of extreme or prolonged ER stress, such as Ca⁺² perturbation agents, apoptotic mediators including transcriptional factor C/EBP homologous protein (CHOP)/GADD153 are increased, caspase-4 is activated and cell death occurs. Studies of the pathogenesis of rotavirus infection suggest that calcium plays an important role in events leading to cell apoptosis. NSP4 is known to play a role in both morphogenesis and pathogenesis of the virus by affecting calcium homeostasis. NSP4 protein significantly increases cytosolic calcium through different mechanisms, including a Phospholipase C (PLC)-dependent mechanism associated with the enterotoxin properties of this protein. However, the most important mechanism is ER degradation mediated by the viroporin properties of this protein and the subsequent increase in cytoplasmic Ca+2 concentration. Some recent studies have shown that rotavirus increases intracellular cytoplasmic Ca+2 levels in insect and mammalian cells through the NSP4 protein. Moreover, similar studies in the related field show that rotavirus induces cell death through apoptosis by this strategy.

It is known from existing studies that an increase in calcium concentration will cause the infection to progress with more viral particles. In similar studies, it has been reported that rotavirus, which requires the presence of sialic acid to bind to and infect cells, is less dependent on the presence of sialic acid on cell surfaces at a calcium concentration of 10 mM, and the reason for this is that high calcium concentration causes conformational changes in the rotavirus particle. Therefore, an average calcium concentration of 10 mM in the diets of individuals in rotavirus susceptible age groups seems to provide ideal conditions for rotavirus replication, thus increasing the likelihood of rotavirus infection and contributing to the formation and spread of infection. In addition, IL-10, as an anti-inflammatory cytokine, may have strong anti-inflammatory effects and IL-10 is also known to play an important role in enteric infections and intestinal inflammation; it is known that diarrhea is more common when TNF-α is high and IL-10 is low, especially in people with irritable bowel syndrome.

In rotavirus infections, non-specific antiviral drugs, various fluid supplements given to alleviate symptoms are among the commonly used clinical treatments. Probiotics are used as alternative methods to treat and/or alleviate the severity of infectious diseases caused by rotavirus infections, including gastroenteritis, due to their potential effects in preventing colonization of pathogens, inhibiting the development of infection and also improving the immunity of patients. However, there is no specific method or component to prevent adverse effects. In the case of vaccines developed for rotavirus, distribution to countries in need is inadequate and the effectiveness of rotavirus vaccines in developing countries, where the disease rate is highest, is very low. In addition, in cases where people have close contact with animals, new strains emerge through genetic transfer between rotavirus strains, which negatively affects immunization.

In a patent document numbered JP2013053089A in the known state of the art, a disease preventive/curative (therapeutic) agent containing certain lactic acid bacteria variant as an active ingredient for a viral disease is disclosed. The prophylactic/therapeutic agent for the prevention of virus infections, including rotavirus infection, and the destruction of virus-infected cells, contains the active ingredient Lactobacillus plantarum AYA variant (accession number FERM P-21106). The lactic acid bacterium Lactobacillus plantarum AYA variant increases IgA production in the intestinal tract and for immunostimulation functions. Similarly, the lactic acid bacterium L-137 increases IL-12 production for immunostimulation functions. The document also explains that the Lactobacillus plantarum AYA variant is effective in destroying virus-infected cells. There is no mention in the document of a pharmaceutical agent that specifically uses the Lactobacillus plantarum bacteriocin PlnA to prevent and/or reduce the adverse effects of the rotavirus NSP4 viral protene in cells and/or organisms.

Another patent document KR101883148B1 of the known art discloses a variant of Lactobacillus plantarum LRC 5310 containing a rotavirus inhibitory/blocking property and an extracellular polysaccharide derived from the variant, and a method of using the variant. The method prevents rotavirus infection by consumption of a food source containing the variant, rather than by vaccination. The document mentions that lactobacillus is an important microorganism added to various fermented foods around the world and that research has shown that drinking milk fermented with lactic acid bacteria or lactic acid bacteria is beneficial in the treatment or prevention of acute diarrhea in children due to rotavirus infection.

There is no mention in the document of a pharmaceutical agent that specifically uses the Lactobacillus plantarum bacteriocin PlnA to prevent and/or reduce the adverse effects of the rotavirus NSP4 viral protene in cells and/or organisms.

Consequently, there is a need for the development of a pharmaceutical agent which prevents and/or eliminates the adverse effects of the rotavirus NSP4 viral protene in cells and/or organisms, and which offers convenience and improvement in terms of production, supply, distribution and therapeutic efficacy.

### PURPOSE OF THE INVENTION

The present invention relates to a pharmaceutical agent that meets the above-mentioned requirements, eliminates possible disadvantages and provides some additional advantages.

The main purpose of the inventive pharmaceutical agent is to provide a pharmaceutical agent that prevents and/or eliminates the adverse effects of the rotavirus NSP4 viral protene in cells and/or organisms.

Another object of the invention is to obtain a pharmaceutical agent suitable for preventive or therapeutic use for planning clinical, dosage, phase and pharmacokinetic studies.

Another object of the invention is to provide a pharmaceutical agent suitable for the use of bacteriocin as an option for the treatment of enteritis due to rotavirus infections.

Another object of the invention is to provide a pharmaceutical agent for the treatment of diseases and infections caused by an increase in intracellular calcium with PlnA, a bacteriocin of Lactobacillus plantarum.

Another object of the invention is to provide a pharmaceutical agent for the prevention and treatment of the adverse effects of rotavirus.

Another object of the invention is to provide a pharmaceutical agent that facilitates and improves production, supply, distribution and treatment efficacy in the prevention and treatment of rotavirus adverse effects.

Another object of the invention is to provide a pharmaceutical agent which enables new approaches in the control and treatment of rotavirus infections.

Another object of the invention is to provide a pharmaceutical agent for inhibiting inflammation induced by NSP4 by reducing the suppression of gene expression of the anti-inflammatory cytokine IL-10.

In order to achieve the above objectives in the most general form, the pharmaceutical agent developed with the present invention, which provides a therapeutic effect that prevents and/or eliminates the adverse effects caused by the Rotavirus NSP4 viral protene in cells and/or organisms, contains the Lactobacillus plantarum bacteriocin Plantaricin A.

The structural and characteristic features and all advantages of the invention will be more clearly understood from the following figures and the detailed description with references to these figures, and therefore the evaluation should be made by taking these figures and the detailed description into consideration.

### DETAILED DESCRIPTION OF THE INVENTION

In this detailed description, the preferred embodiments of the inventive pharmaceutical agent are described only for the purpose of a better understanding of the subject matter.

The pharmaceutical agent developed with the present invention, which provides a therapeutic effect by preventing and/or eliminating the adverse effects of the rotavirus NSP4 viral protene in the cell and/or organism, preferably increasing the protein level of the anti-inflammatory cytokine IL-10, preferably increasing the gene expression level of the anti-inflammatory cytokine IL-10, preferably increasing the pro-inflammatory cytokine levels, preferably increasing the pro-inflammatory cytokine levels, comprises the Lactobacillus plantarum bacteriocin Plantaricin A (PlnA).

In an exemplary embodiment of the pharmaceutical agent developed with the present invention, a pharmaceutical agent containing the Lactobacillus plantarum bacteriocin Plantaricin A (PlnA) is used to reduce the intracellular calcium content of the rotavirus NSP4 protein in the intestinal cell line. Thus, the amount of intracellular calcium increased by the NSP4 protein is reduced and, therefore, cell death and ER disruption caused by rotavirus through apoptosis is prevented by this strategy.

For example, the most significant difference in the amount of intracellular calcium (Ca) with PlnA-containing pharmaceutical agent administration occurs at the 6th hour after administration. The rate of increase in intracellular calcium in the first NSP4-treated group was 2.27 times higher than the control group without NSP4 at 6 hours. The rate of increase in the amount of intracellular calcium in the first NSP4-treated group was 2.27 times higher than the control group without NSP4 at 6 hours. In the case of co-administration of NSP4 and PlnA-containing pharmaceutical agent, the amount of calcium in the 6th hour decreased 3.75-fold compared to the NSP4-treated first group and 1.64-fold compared to the control group. Thus, by increasing the membrane permeability of PlnA, the post-replication maturation and dissemination stages of the virus in rotaviral infections are prevented (Graph 1).

In addition, during the observation of longer-term effects of the pharmaceutical agent, the rate of increase in intracellular calcium in the first NSP4-treated group was 2.43 times higher than in the control group without NSP4 at 12 hours. In the case of co-administration of NSP4 and PlnA-containing pharmaceutical agent, the amount of calcium at 12 hours decreased 2.67 times compared to the first group administered NSP4 and the same amount of calcium was observed in the control group (Graph 2).

The protein levels of IL-10, which has an important role in enteric infections and intestinal inflammation and increases the likelihood of diarrhea in people with irritable bowel syndrome if it is low, are examined. Compared to the control group without any application, the protein levels in the group administered with NSP4 and PlnA-containing pharmaceutical agent increased 2-fold at the 3rd hour and 1.43-fold at the 6th hour (Graph 3).

While no significant difference was observed at 12 hours after the application, a 1.13-fold increase in the protein levels of IL-10 was again obtained at 24 hours (Graph 4).

Furthermore, when the mRNA levels of IL-10 were analyzed, the gene expression of IL-10 was suppressed 1.65-fold in the first group administered NSP4 compared to the control group without any treatment, but this suppression was reduced in the group administered NSP4 and PlnA containing pharmaceutical agent together. Even at the 12th hour, the mRNA level of IL-10 increases 0.68-fold compared to the control group (Graph 5). In this way, the inflammation induced by NSP4 is inhibited and both protein and gene expression levels of IL-10 are increased.

The pharmaceutical agent developed by the present invention prevents and eliminates the negative effects of the rotavirus NSP4 viral protene in cells and/or organisms.

PlnA effectively reduces the calcium levels increased inside and outside the cell by NSP4 and inhibits the inflammation caused by NSP4 by increasing both protein and gene expression levels of IL-10, which has strong anti-inflammatory effects. In this way, a pharmaceutical agent with high protection and treatment efficacy against the adverse effects of rotavirus and rotavirus infections, reducing costs for different protection and treatment applications, offering convenience and improvement in terms of production, supply, distribution and treatment efficacy is obtained.

1. A pharmaceutical agent that prevents and/or eliminates the adverse effects caused by the rotavirus NSP4 viral protene in the cell and/or organism and offers therapeutic effect, the pharmaceutical agent comprising:
Lactobacillus plantarum bacteriocin comprising Plantarisin A.

2. A pharmaceutical agent that prevents and/or eliminates the negative effects of the rotavirus NSP4 viral protene in cells and/or organisms and increases the protein level of the anti-inflammatory cytokine IL-10, the pharmaceutical agent comprising Lactobacillus plantarum bacteriocin Plantaricin A.

3. A pharmaceutical agent that prevents and/or eliminates the negative effects of the rotavirus NSP4 viral protene in cells and/or organisms and increases the gen expression level of the anti-inflammatory cytokine IL-10, the pharmaceutical agent comprising Lactobacillus plantarum bacteriocin Plantaricin A.

4. A pharmaceutical agent that prevents and/or eliminates the adverse effects of rotavirus NSP4 viral protene in cells and/or organisms and increases proinflammatory cytokine levels, the pharmaceutical agent comprising Lactobacillus plantarum bacteriocin Plantaricin A.
